(19) United States
(12) Patent Application Publication
(10) Pub. No.: US 2006/0030581 A1
(43) Pub. Date: Feb. 9, 2006

(54) Mannitol formulation for integrin receptor antagonist

(76) Inventors: Laura A. DeBusi, Schwenksville, PA (US); Sami Karaborni, Lansdale, PA (US); Bozen K. Matuszewska, North Wales, PA (US); Adama T. Procopio, Cinnaminson, PA (US); Wei Xu, North Wales, PA (US); Chia-Yi Yang, North Wales, PA (US)

Correspondence Address: MERCK AND CO., INC, P O BOX 2000, RAHWAY, NJ, 07065-0907, US

(21) Appl. No.: 10/527,605

(22) PCT Filed: Sep. 16, 2003
(86) PCT No.: PCT/US03/28959

§ 371 Date: Mar. 14, 2005

Related U.S. Application Data
(60) Provisional application No. 60/412,262, filed on Sep. 20, 2002.

(51) Int. Cl. A61K 31/4745

(52) U.S. Cl. 514/300

(57) ABSTRACT

Disclosed are pharmaceutical compositions of an integrin αvβ3 receptor antagonist containing mannitol as the binding agent. The compositions are prepared by wet granulation or direct compression tablet formulation. These pharmaceutical formulations are useful for inhibiting bone resorption associated with osteoporosis, metastatic bone disease, hypercalcemia of malignancy, and Paget's disease.

### FIELD OF THE INVENTION

The present invention is directed to novel pharmaceutical compositions containing the integrin αvβ3 receptor antagonist 3-{2-oxo-3-[3-(5,6,7,8-tetrahydro-[1,8]naphthyridin-2-yl)-propyl]imidazolidin-1-yl}-3(S)-(6-methoxypyridin-3-yl)propionic acid, or a pharmaceutically acceptable salt thereof, methods of preparing such pharmaceutical compositions, and methods of inhibiting bone resorption and treating osteoporosis with such pharmaceutical compositions.

### BACKGROUND OF THE INVENTION

The compound 3-{2-oxo-3-[3-(5,6,7,8-tetrahydro-[1,8]naphthyridin-2-yl)-propyl]imidazolidin-1-yl}-3(S)-(6-methoxypyridin-3-yl)propionic acid of structural formula I (Compound I), or a pharmaceutically acceptable salt thereof, is disclosed in U.S. Pat. No. 6,017,926 (Jan. 25, 2000). Compound I is an antagonist of the integrin αvβ3 receptor and is useful for inhibiting bone resorption, restenosis, angiogenesis, diabetic retinopathy, macular degeneration, inflammatory arthritis, cancer, and metastatic tumor growth. It is particularly useful for inhibiting bone resorption associated with osteoporosis, metastatic bone disease, hypercalcemia of malignancy, and Paget's disease.

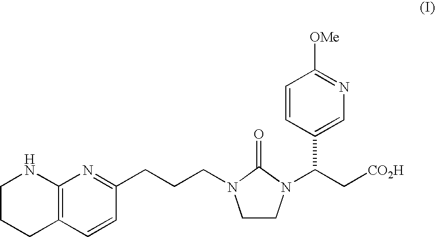

Pharmaceutical compositions containing Compound I in the dosage form of compressed tablets can be prepared by either wet granulation or direct compression techniques. Tablets prepared by wet granulation usually require the addition of a diluent to form granules. The diluent when contacted with water will swell or start to dissolve to form a gel-like consistency. The wet formulation process helps to form agglomerates of powders. These agglomerates are called “granules.” Typical diluents include starch, starch paste, gelatin, and cellulosics, such as hydroxypropylmethyl cellulose (HPMC), hydroxyethyl cellulose, hydroxypropylcellulose (HPC), and polyvinyl pyrrolidone (See, Remington's Pharmaceutical Sciences, 18^{th} ed., pp 1635-1636). Microcrystalline cellulose functions primarily as a bulking agent in wet granulation formulations. The use of microcrystalline cellulose in wet granulation or direct compression tablet formulations of Compound I results in physical instability of stressed tablets, such as tablets exposed to elevated humidities and temperatures.

The present invention provides for pharmaceutical compositions of Compound I prepared by wet granulation or direct compression with mannitol or a mannitol/microcrystalline cellulose mixture as the diluent. The use of mannitol or a mannitol/microcrystalline cellulose mixture in place of microcrystalline cellulose alone results in greater physical stability of coated and uncoated tablets containing Compound I to elevated humidities and temperatures.

The present invention also provides a process to prepare pharmaceutical compositions of Compound I by wet granulation or direct compression methods using mannitol or a mannitol/microcrystalline cellulose mixture as the diluent.

Another object of the present invention provides methods for inhibiting bone resorption and treating osteoporosis by administering to a host in need of such treatment a therapeutically effective amount of one of the mannitol-based pharmaceutical compositions of the present invention.

These and other objects will become readily apparent from the detailed description which follows.

### DESCRIPTION OF THE INVENTION

One aspect of the present invention is directed to solid dosage forms, particularly tablets, for the medicinal administration of Compound I or a pharmaceutically acceptable salt thereof.

In a particular aspect of the present invention, the tablets comprise (a) Compound I, or a pharmaceutically acceptable salt thereof, as the active pharmaceutical ingredient, (b) mannitol or a mannitol/microcrystalline cellulose mixture as the diluent, (c) a disintegrant, and (d) a lubricant. In a class of this embodiment, the tablet may also contain a binding agent and/or an antioxidant. Finally, the tablet may be film-coated to provide additional stability to the dosage form, and a colorant, sweetening agent, and/or flavoring agent may be added if desired.

Mannitol or a mixture of mannitol and microcrystalline cellulose functions as a diluent in the wet granulation or direct compression method for the preparation of tablet formulations of Compound I.

The disintegrant may be one of several modified starches or modified cellulose polymers. In one embodiment, the disintegrant is croscarmellose sodium. Croscarmellose sodium NF Type A is commercially available under the trade name “Ac-di-sol.”

Embodiments of lubricants include magnesium stearate, calcium stearate, stearic acid, surface active agents such as sodium lauryl sulfate, propylene glycol, sodium dodecanesulfonate, sodium oleate sulfonate, and sodium laurate mixed with stearates and talc, sodium stearyl fumarate, and other known lubricants. In one class of these embodiments, the lubricant is magnesium stearate.

The tablets of the present invention may optionally contain a binding agent selected from the group consisting of hydroxypropylcellulose (HPC), hydroxypropylmethyl cellulose (HMPC), hydroxyethyl cellulose, and polyvinyl pyrrolidone. One embodiment of such a binding agent is hydroxypropylcellulose.

An antioxidant may optionally be added to the formulation to impart chemical stability. The antioxidant is selected from the group consisting of α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), and butylated hydroxyanisole (BHA). In one embodiment, the antioxidant is BHT or BHA.

Finally, the compressed tablets may be film-coated such as with a mixture of hydroxypropylcellulose and hydroxypropylmethylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; or any other suitable immediate-release film-coating agent(s).

Methods for the preparation of Compound I are disclosed in U.S. Pat. No. 6,017,926 (Jan. 25, 2000), the contents of which are incorporated by reference in their entirety, and in WO 01/34602 (17 May 2001). A pharmaceutically acceptable salt of Compound I may also be employed in the present invention.

In one embodiment the pharmaceutical compositions of the present invention are prepared by wet granulation methods and comprise about 25 to 70% by weight of Compound I as the active ingredient; about 25 to 70% by weight of mannitol or a mixture of mannitol and microcrystalline cellulose; about 1 to 5% by weight of a disintegrant; about 0 to 5% by weight of a binding agent; and about 1 to 3% by weight of a lubricant. In a class of this embodiment the disintegrant is croscarmellose sodium, the binding agent is hydroxypropylcellulose, and the lubricant is magnesium stearate. In a subclass of this class, the pharmaceutical compositions comprise about 33 to 67% by weight of Compound I as the active ingredient; about 25-60% by weight of mannitol; about 1 to 4% by weight of croscarmellose sodium; about 1 to 4% by weight of hydroxypropylcellulose; and about 1 to 2% by weight of magnesium stearate. In a further subclass of this class of this embodiment the pharmaceutical compositions comprise about 33 to 67% by weight of Compound I as the active ingredient; about 25-60% by weight of mannitol; about 3% by weight of croscarmellose sodium; about 3% by weight of hydroxypropylcellulose; and about 2% by weight of magnesium stearate.

The pharmaceutical compositions may optionally comprise about 0 to 0.2% by weight of an antioxidant, such as BHT and BHA. In one embodiment the pharmaceutical composition comprises about 0.02% by weight of an antioxidant.

In another embodiment the pharmaceutical compositions of the present invention are prepared by direct compression methods and comprise about 33 to 67% by weight of Compound I as the active pharmaceutical ingredient; about 25 to 60% by weight of mannitol; about 1 to 5% by weight of a disintegrant; about 0 to 20% by weight of microcrystalline cellulose; about 0 to 5% by weight of a binding agent; and about 1 to 3% by weight of a lubricant. In a class of this embodiment the disintegrant is croscarmellose sodium, the binding agent is hydroxypropylcellulose, and the lubricant is magnesium stearate. In a subclass of this class the pharmaceutical compositions comprise about 33 to 67% by weight of Compound I as the active pharmaceutical ingredient; about 25 to 60% by weight of mannitol; about 3% by weight of croscarmellose sodium; about 3% by weight of hydroxypropylcellulose; and about 2% by weight of magnesium stearate.

In another embodiment the pharmaceutical compositions are generally in the form of compressed tablets. The tablets may be, for example, from 50 mg to 800 mg net weight. In a class of this embodiment, the tablets may be from 75 mg to 700 mg net weight. In a subclass of this class, the tablets may be from 100 mg to 600 mg net weight. The potency of the active pharmaceutical ingredient (Compound I) in these tablets is about 50 to 400 mg.

In a further embodiment of the present invention, the pharmaceutical compositions as envisioned for commercial development are as follows:

Tablets of 50 mg Potency Compound I (about 33% Drug Loading):

About 33% by weight of Compound I; about 40% by weight of mannitol; about 20% by weight of microcrystalline cellulose; about 3% by weight of croscarmellose sodium; about 2% by weight of magnesium stearate; about 3% by weight of hydroxypropylcellulose (HPC); and optionally about 0.02% by weight of BHT or BHA. In the absence of microcrystalline cellulose, about 33% by weight of Compound I; about 60% by weight of mannitol; about 3% by weight of croscarmellose sodium; about 2% by weight of magnesium stearate; about 3% by weight of hydroxypropylcellulose (HPC); and optionally about 0.02% by weight of BHT or BHA.

Tablets of 50 mg Potency Compound I (about 50% Drug Loading):

About 50% by weight of Compound I; about 40% by weight of mannitol; about 3% by weight of croscarmellose sodium; about 2% by weight of magnesium stearate; about 3% by weight of hydroxypropylcellulose (HPC); and optionally about 0.03% by weight of BHT or BHA.

Tablets of 100 mg Potency of Compound I (about 67% Drug Loading):

About 67% by weight of Compound I; about 25% by weight of mannitol; about 3% by weight of croscarmellose sodium; about 2% by weight of magnesium stearate; about 3% by weight of hydroxypropylcellulose (HPC); and optionally about 0.02% by weight of BHT or BHA.

Tablets of 200 mg Potency Compound I (about 67% Drug Loading):

About 67% by weight of Compound I; about 25% by weight of mannitol; about 3% by weight of croscarmellose sodium; about 2% by weight of magnesium stearate; about 3% by weight of hydroxypropylcellulose (HPC); and optionally about 0.02% by weight of BHT or BHA.

Tablets of 400 mg Potency Compound I (about 67% Drug Loading):

About 67% by weight of Compound I; about 25% by weight of mannitol; about 3% by weight of croscarmellose sodium; about 2% by weight of magnesium stearate; about 3% by weight of hydroxypropylcellulose (HPC); and optionally about 0.02% by weight of BHT or BHA.

In one embodiment of the present invention, each of the tablets of the potencies above is formulated in a 100 mg, 150 mg, 300 mg, or 600 mg tablet. The tablets are optionally film-coated with about 4% by weight of HPC/HPMC film-coat or about 4% by weight of polyvinyl alcohol (PVA)/polyethylene glycol (PEG) film-coat, each containing titanium dioxide.

The pharmaceutical tablet compositions of the present invention may also contain one or more additional formulation ingredients selected from a wide variety of excipients known in the pharmaceutical formulation art. According to the desired properties of the tablet, any number of ingredients may be selected, alone or in combination, based upon their known uses in preparing tablet compositions. Such ingredients include, but are not limited to, diluents, compression aids, disintegrants, lubricants, flavors, flavor enhancers, sweeteners, and preservatives.

The term “tablet” as used herein is intended to encompass compressed pharmaceutical dosage formulations of all shapes and sizes, whether coated or uncoated. Substances which may be used for coating include hydroxypropylcellulose, hydroxypropylmethylcellulose, titanium dioxide, talc, sweeteners, colorants, and flavoring agents.

The tablet formulations of the present invention are prepared either by wet granulation or direct compression methods. The steps involved in the wet-granulation method comprise:
(1) adding Compound I, mannitol, microcrystalline cellulose (optional), croscarmellose sodium, and hydroxypropyl cellulose to the bowl of the granulator and dry mixing for a period of about 1 min to 3 min;
(2) adding the appropriate granulating solution over a period of about 2 to 5 min; for formulations containing BHA or BHT, the granulating solution comprises about 15-18% ethanol: 85-82% water is utilized; for formulations without BHA or BHT, an aqueous granulating solution is used; a total of about 35-50% (w/w) granulating solution is added to the batch;
(3) wet massing for a period of about 0 to 5 min;
(4) drying in an oven or fluid bed dryer to remove water; the oven temperature is about 40° C. or the fluid bed dryer inlet temperature is about 55° C. and the drying is performed for about 12 h for oven drying or 0.5 to 1 h for fluid bed drying;
(5) milling;
(6) lubricating (such as with magnesium stearate);
(7) compressing the lubricated granule mixture into a desired tablet image; and, if desired,
(8) film-coating.

Granulation is the process of adding the water to a powder mixture with mixing until granules are formed. The granulation step may be varied from 1 to 15 min, preferably 2 to 5 min. The lubrication step is the process of adding lubricant to the mixture; the lubrication step may be varied from 1 to 15 min, preferably 2 to 5 min.

The steps involved in the direct compression method comprise:
(1) blending Compound I, mannitol, and croscarmellose sodium in a V-blender or other suitable blender for a period of about 5 to 30 min;
(2) optionally adding hydroxypropyl cellulose and/or microcrystalline cellulose to improve compaction properties;
(3) lubricating (such as with magnesium stearate) for about 1 to 15 min;
(4) compressing the lubricated blend into a desired tablet image; and, if desired,
(5) film-coating.

An antioxidant, such as BHA or BHT, can be added by either layering it onto one of the excipients, preferably the mannitol, prior to blending with Compound I and the other excipients or by layering it onto Compound I during the bulk drug synthesis process.

The present invention provides methods for inhibiting bone resorption and treating osteoporosis by orally administering to a host in need of such treatment a therapeutically effective amount of one of the mannitol-based pharmaceutical compositions of the present invention. In one embodiment the host in need of such treatment is a human. In another embodiment the pharmaceutical composition is in the dosage form of a tablet.

The following examples further describe and demonstrate embodiments within the scope of the present invention. The examples are given solely for the purpose of illustration and are not intended to be construed as limitations of the present invention as many variations thereof are possible without departing from the spirit and scope of the invention.

### EXAMPLE 1

100 mg Potency Tablet of Compound I
(67% drug loading) - wet granulation

| | | |
|---|---|---|
| Compound I* | 101.1 | mg |
| Mannitol** | 36.87 | mg |
| Hydroxypropyl cellulose | 4.50 | mg |
| Croscarmellose sodium | 4.50 | mg |
| Magnesium stearate | 3.00 | mg |
| Butylated Hydroxyanisole (BHA) | 0.03 | mg |

*Equivalent to 100 mg of the anhydrate.
**Weight adjusted to account for water and impurities in the API.

Method of Manufacture:

The active pharmaceutical ingredient (Compound I), mannitol (Roquette 35), croscarmellose sodium, and hydroxypropyl cellulose (Kucel EXF) were dry mixed using a high-shear granulator for 2 min. The granulating solvent (30 to 45% of a mixture of 82% purified water and 18% ethyl alcohol, in which the BHA was dissolved) was added to this blend with the high-shear granulator running over a 3 min period. The wetted mass was dried in a fluid-bed dryer at an inlet temperature of 55° C. for 0.5 to 1 h. The dried material was then milled using a cone mill to achieve fine granules. After milling, magnesium stearate (lubricant) was added to the blend using a twin-shell blender for a period of 3 min. The lubricated mixture was compressed using a rotary tablet press to provide a 150.0 mg tablet containing 100 mg of active ingredient. The tablet was optionally film-coated with 6.00 mg of a standard HPC/HPMC/TiO₂ film-coat formula to provide a 156.0 mg coated tablet. Any suitable colorant, including lakes or dyes, may be added to the film coat to impart the desired color, in the range of 0-1% tablet weight.

### EXAMPLE 2

200 mg Potency Tablet of Compound I
(67% drug loading) - wet granulation

| | | |
|---|---|---|
| Compound I* | 202.2 | mg |
| Mannitol** | 73.74 | mg |
| Hydroxypropyl cellulose | 9.00 | mg |
| Croscarmellose sodium | 9.00 | mg |
| Magnesium stearate | 6.00 | mg |
| Butylated Hydroxyanisole (BHA) | 0.06 | mg |

*Equivalent to 200 mg of the anhydrate.
**Weight adjusted to account for water and impurities in the API.

Method of Manufacture:

Tablets were prepared using essentially the procedure of Example 1 to provide a 300.0 mg tablet containing 200 mg of active ingredient. The tablets were optionally coated with 12.00 mg of a standard HPC/HPMC/TiO₂ film-coat formula (Opadry I®) to provide a 312.0 mg coated tablet.

### EXAMPLE 3

400 mg Potency Tablet of Compound I
(67% drug loading) - wet granulation

| | | |
|---|---|---|
| Compound I* | 404.4 | mg |
| Mannitol** | 147.48 | mg |
| Hydroxypropyl cellulose | 18.00 | mg |
| Croscarmellose sodium | 18.00 | mg |
| Magnesium stearate | 12.00 | mg |
| Butylated Hydroxyanisole (BHA) | 0.12 | mg |

*Equivalent to 200 mg of the anhydrate.
**Weight adjusted to account for water and impurities in the API.

Method of Manufacture:

Tablets were prepared using essentially the procedure of Example 1 to provide a 600.0 mg tablet containing 400 mg of active ingredient. The tablets were optionally coated with 24.00 mg of a standard HPC/HPMC/TiO₂ film-coat formula (Opadry I®) to provide a 624.0 mg coated tablet.

### EXAMPLE 4

50 mg Potency Tablet of Compound I
(33% drug loading) - wet granulation

| | | |
|---|---|---|
| Compound I* | 51.00 | mg |
| Mannitol** | 58.48 | mg |
| Microcrystalline Cellulose (Avicel) | 29.24 | mg |
| Hydroxypropyl cellulose | 4.50 | mg |
| Croscarmellose sodium | 4.50 | mg |
| Magnesium stearate | 2.25 | mg |
| Butylated Hydroxyanisole (BHA) | 0.030 | mg |

*Equivalent to 50 mg of the anhydrate.
**Weight adjusted to account for water and impurities in the API.

Method of Manufacture:

Tablets were prepared using essentially the procedure of Example 1 to provide a 150.0 mg tablet containing 50 mg of active ingredient. The tablets were optionally coated with 6.00 mg of a standard HPC/HPMC/TiO₂ film-coat formula (Opadry I®) to provide a 156.0 mg coated tablet.

### EXAMPLE 5

50 mg Potency Tablet of Compound I
(50% drug loading) - wet granulation

| | | |
|---|---|---|
| Compound I* | 51.07 | mg |
| Mannitol** | 40.9 | mg |
| Hydroxypropyl cellulose | 3.00 | mg |
| Croscarmellose sodium | 3.00 | mg |
| Magnesium stearate | 2.00 | mg |
| Butylated Hydroxyanisole (BHA) | 0.030 | mg |

*Equivalent to 50 mg of the anhydrate.
**Weight adjusted to account for water and impurities in the API.

Method of Manufacture:

Tablets were prepared using essentially the procedure of Example 1 to provide a 100.0 mg tablet containing 50 mg of active ingredient. The tablets were optionally coated with 4.00 mg of a standard HPC/HPMC/TiO₂ film-coat formula (Opadry I®) to provide a 104.0 mg coated tablet.

### EXAMPLE 6

50 mg Potency Tablet of Compound I
(33% drug loading) - wet granulation

| | | |
|---|---|---|
| Compound I* | 51.0 | mg |
| Mannitol** | 86.22 | mg |
| Hydroxypropyl cellulose | 4.50 | mg |
| Croscarmellose sodium | 4.50 | mg |
| Magnesium stearate | 3.75 | mg |
| Butylated Hydroxyanisole (BHA) | 0.030 | mg |

*Equivalent to 50 mg of the anhydrate.
**Weight adjusted to account for water and impurities in the API.

Method of Manufacture:

Tablets were prepared using essentially the procedure of Example 1 to provide a 150.0 mg tablet containing 50 mg of active ingredient. The tablets were optionally coated with 6.00 mg of a standard HPC/HPMC/TiO₂ film-coat formula (Opadry I®) to provide a 156.0 mg coated tablet.

### EXAMPLE 7

50 mg Potency Tablet of Compound I
(33% drug loading) - direct compression

| | | |
|---|---|---|
| Compound I* | 51.0 | mg |
| Mannitol** | 90.75 | mg |
| Croscarmellose sodium | 4.50 | mg |
| Magnesium stearate | 3.75 | mg |

*Equivalent to 50 mg of the anhydrate.
**Weight adjusted to account for water and impurities in the API.

Method of Manufacture:

Tablets were prepared using the direct compression procedure described above to provide a 150.0 mg tablet containing 50 mg of active ingredient.

### EXAMPLE 8

50 mg Potency Tablet of Compound I
(33% drug loading) - direct compression

| | | |
|---|---|---|
| Compound I* | 51.0 | mg |
| Mannitol** | 60.5 | mg |
| Microcrystalline cellulose | 30.25 | mg |
| Croscarmellose sodium | 4.50 | mg |
| Magnesium stearate | 3.75 | mg |

*Equivalent to 50 mg of the anhydrate.
**Weight adjusted to account for water and impurities in the API.

Method of Manufacture:

Tablets were prepared using the direct compression procedure described above to provide a 150.0 mg tablet containing 50 mg of active ingredient.

### EXAMPLE 9

50 mg Potency Tablet of Compound I
(50% drug loading) - direct compression

| | | |
|---|---|---|
| Compound I* | 51.0 | mg |
| Mannitol** | 44 | mg |
| Croscarmellose sodium | 3.0 | mg |
| Magnesium stearate | 2.0 | mg |

*Equivalent to 50 mg of the anhydrate.
**Weight adjusted to account for water and impurities in the API.

Method of Manufacture:

Tablets were prepared using the direct compression procedure described above to provide a 100.0 mg tablet containing 50 mg of active ingredient.

### EXAMPLE 10

100 mg Potency Tablet of Compound I
(67% drug loading) - direct compression

| | | |
|---|---|---|
| Compound I* | 101.1 | mg |
| Mannitol** | 41.4 | mg |
| Croscarmellose sodium | 4.50 | mg |
| Magnesium stearate | 3.0 | mg |

*Equivalent to 100 mg of the anhydrate.
**Weight adjusted to account for water and impurities in the API.

Method of Manufacture:

Tablets were prepared using the direct compression procedure described above to provide a 150.0 mg tablet containing 100 mg of active ingredient.

### EXAMPLE 11

200 mg Potency Tablet of Compound I (67%
drug loading) - direct compression

| | | |
|---|---|---|
| Compound I* | 202.2 | mg |
| Mannitol** | 82.8 | mg |
| Croscarmellose sodium | 9.0 | mg |
| Magnesium stearate | 6.0 | mg |

*Equivalent to 200 mg of the anhydrate.
**Weight adjusted to account for water and impurities in the API.

Method of Manufacture:

Tablets were prepared using the direct compression procedure described above to provide a 300.0 mg tablet containing 200 mg of active ingredient.

### EXAMPLE 12

400 mg Potency Tablet of Compound I (67%
drug loading) - direct compression

| | | |
|---|---|---|
| Compound I* | 404.4 | mg |
| Mannitol** | 165.6 | mg |
| Croscarmellose sodium | 18.0 | mg |
| Magnesium stearate | 12.0 | mg |

*Equivalent to 400 mg of the anhydrate.
**Weight adjusted to account for water and impurities in the API.

Method of Manufacture:

Tablets were prepared using the direct compression procedure described above to provide a 600.0 mg tablet containing 400 mg of active ingredient.

While the foregoing specification teaches the principles of the present invention, with examples provided for the purpose of illustration, it will be understood that the practice of the invention encompasses all of the casual variations, adaptations, modifications, deletions, or additions of procedures and protocols described herein, as come within the scope of the following claims and equivalents.

What is claimed is:

1. A pharmaceutical composition comprising about 25 to 70% by weight of the active ingredient of structural formula I 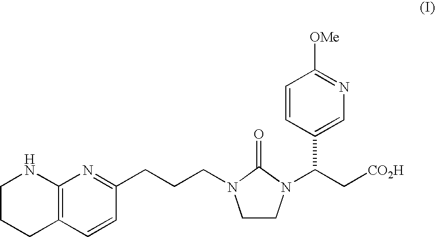
or a pharmaceutically acceptable salt thereof;
about 25 to 70% by weight of mannitol or about 25 to 70% by weight of a mixture of mannitol and microcrystalline cellulose;
about 1 to 5% by weight of a disintegrant;
about 0 to 5% by weight of a binding agent; and
about 1 to 3% by weight of a lubricant.

2. The pharmaceutical composition of claim 1 wherein said disintegrant is croscarmellose sodium, said binding agent is hydroxypropylcellulose, and said lubricant is magnesium stearate.

3. The pharmaceutical composition of claim 2 comprising
about 33 to 67% by weight of said active ingredient;
about 25 to 60% by weight of mannitol;
about 1 to 4% by weight of croscarmellose sodium;
about 1 to 4% by weight of hydroxypropylcellulose; and
about 1 to 2% by weight of magnesium stearate.

4. The pharmaceutical composition of claim 3 comprising
about 33 to 67% by weight of said active ingredient;
about 25 to 60% by weight of mannitol;
about 3% by weight of croscarmellose sodium;
about 3% by weight of hydroxypropylcellulose; and
about 2% by weight of magnesium stearate.

5. The pharmaceutical composition of claim 1 additionally comprising about 0 to 0.2% by weight of an antioxidant.

6. The pharmaceutical composition of claim 5 wherein said antioxidant is BHT or BHA.

7. The pharmaceutical composition of claim 2 comprising
about 33% by weight of said active ingredient;
about 60% by weight of mannitol;
about 3% by weight of croscarmellose sodium;
about 3% by weight of hydroxypropylcellulose; and
about 2% by weight of magnesium stearate.

8. The pharmaceutical composition of claim 7 additionally comprising about 0.02% by weight of BHT or BHA.

9. The pharmaceutical composition of claim 2 comprising
about 33% by weight of said active ingredient;
about 40% by weight of mannitol;
about 20% by weight of microcrystalline cellulose;
about 3% by weight of croscarmellose sodium;
about 3% by weight of hydroxypropylcellulose; and
about 2% by weight of magnesium stearate.

10. The pharmaceutical composition of claim 9 additionally comprising about 0.02% by weight of BHT or BHA.

11. The pharmaceutical composition of claim 2 comprising
about 50% by weight of said active ingredient;
about 40% by weight of mannitol;
about 3% by weight of croscarmellose sodium;
about 3% by weight of hydroxypropylcellulose; and
about 2% by weight of magnesium stearate.

12. The pharmaceutical composiion of claim 11 additionally comprising about 0.02 to 0.03% by weight of BHT or BHA.

13. The pharmaceutical composition of claim 2 comprising
about 67% by weight of said active ingredient;
about 25% by weight of mannitol;
about 3% by weight of croscarmellose sodium;
about 3% by weight of hydroxypropylcellulose; and
about 2% by weight of magnesium stearate.

14. The pharmaceutical composition of claim 13 additionally comprising about 0.02% by weight of BHT or BHA.

15. The pharmaceutical composition of claim 1 prepared by wet granulation methods.

16. A pharmaceutical composition comprising about 33 to 67% by weight of the active ingredient of structural formula I 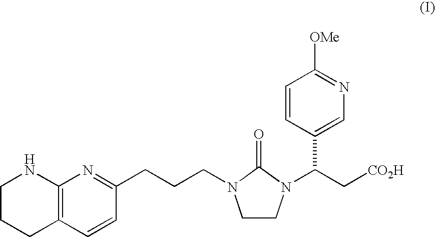
or a pharmaceutically acceptable salt thereof;
about 25 to 60% by weight of mannitol;
about 0 to 20% by weight of microcrystalline cellulose;
about 1 to 5% by weight of a disintegrant;
about 0 to 5% by weight of a binding agent; and
about 1 to 3% by weight of a lubricant.

17. The pharmaceutical composition of claim 16 wherein said disintegrant is croscarmellose sodium, said binding agent is hydroxypropylcellulose, and said lubricant is magnesium stearate.

18. The pharmaceutical composition of claim 17 comprising
about 33 to 67% by weight of said active ingredient;
about 25 to 60% by weight of mannitol;
about 3% by weight of croscarmellose sodium;
about 3% by weight of hydroxypropylcellulose; and
about 2% by weight of magnesium stearate.

19. The pharmaceutical composition of claim 16 prepared by direct compression methods.

20. A method of inhibiting bone resorption in a human in need thereof comprising orally administering to said human a bone resorption-inhibitory amount of the pharmaceutical composition of claim 1.

21. A method of treating osteoporosis in a human in need thereof comprising orally administering to said human a therapeutically effective amounnt of the pharmaceutical composition of claim 1.

22. The pharmaceutical composition of claim 1 further comprising one or more agents selected from the group consisting of flavoring agents, colorants, and sweeteners.
